(19) United States
(12) Patent Application Publication
(10) Pub. No.: US 2009/0069767 A1
(43) Pub. Date: Mar. 12, 2009

(54) HAEMOSTATIC DEVICE FOR MINIMALLY INVASIVE SURGERY

(75) Inventors: Erich Odermatt, Schaffhausen (CH); Juergen Wegmann, Stockach (DE); Bernd Blender, Hohentengen (DE)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: AESCULAP AG, a corporation of Germeny, Tuttlingen/Donau (DE)

(21) Appl. No.: 12/174,653

(22) Filed: Jul. 17, 2008

(30) Foreign Application Priority Data
Jul. 24, 2007 (DE) DE 102007037053.0

(51) Int. Cl. A61F 13/551; A61F 13/53; A61F 13/45

(52) U.S. Cl. 604/374; 604/385.01; 604/385.02

(57) ABSTRACT

A haemostatic device for minimally invasive surgery, especially that performed in the abdominal cavity, is in the form of an absorbent disc made of a biodegradable porous material with a center and a radial extension of at least 10 mm from the center, where the disc is formed in such a way that it can be converted into a radially reduced intermediate state with an essentially rotation-symmetrically shortened radius, in which state it can be introduced into the body through a tube, from which it can be released in the flat form.

### RELATED APPLICATION

This application claims priority of German Patent Application No. 102007037053.0, filed Jul. 24, 2007, herein incorporated by reference.

### TECHNICAL FIELD

This disclosure relates to a haemostatic device for minimally invasive surgery, especially that perforned in the abdominal cavity.

### BACKGROUND

Numerous different methods can be used for haemostasis. However, the possibilities of haemostasis for minimally invasive surgery are limited. Fibrin adhesives are often used for this purpose. However, the danger here is that, in the case of heavy bleeding, the adhesives are flushed out of the wound area before the wound is sufficiently sealed and the bleeding stops.

Haemostatic fleeces have also been found suitable for haemostasis and are successful products available on the market. These haemostatic products have a fleece-like or foam-like structure and generally consist of collagen or gelatin. They have a high absorption capacity. In the case of open surgery, these fleeces are lightly pressed on the wound by hand until the bleeding stops. If the bleeding is heavy, a number of fleece layers need to be applied to the wound in some cases. However, it is difficult to use these fleeces in the case of minimally invasive surgery, since they should be rolled up and inserted with the aid of a trocar. They should then be unrolled again in the abdominal cavity with the aid of holding forceps. This requires great skill on the part of the surgeon. The manipulation involved is made even more difficult by the fact that the rolled-up fleeces readily stick together when they come into contact with body fluids, so they cannot be unrolled any more. Such “patches,” based on collagen, are described in EP 1,368,419 B1 and EP 1,343,542 B1. They additionally contain a mixture of fibrinogen and thrombin to enable them to act as fibrin adhesives at the same time.

To avoid having to unroll such fleece or foam pieces, we conducted internal experiments with small pieces of fleece having a surface area that fitted into the inside cross-section of a trocar. These tablet-shaped fleece pieces were easy to dispense and position, but their haemostatic effect was unsatisfactory, because gaps were necessarily present between the individual pieces. Therefore, we continued the search for other ways of obtaining good haemostatic results, while ensuring an easy placement at the same time.

### SUMMARY

We provide a haemostatic device for minimally invasive surgery including an absorbent disc made of a biodegradable porous material with a center and a radial extension of at least about 10 mm from the center, the disc formed to be converted into a radially reduced intermediate state with an essentially rotation-symmetrically shortened radius, in which state it can be introduced into a body through a tube, from which it can be released in a flat form.

### BRIEF DESCRIPTION OF THE DRAWINGS

Further characteristics and advantages of our devices will emerge from the following description of representative structures, based on the drawings and examples.

In the drawings:

FIG. 1 shows a top plan view of a haemostatic device in a flat state;

FIG. 2 shows a perspective view of a device of FIG. 1 when folded to obtain a cup-shaped object;

FIG. 3 shows a partial sectional view of another device in a multiple form in a holder;

FIG. 4 shows a partial sectional view of a further device in a multiple form in a holder; and

FIG. 5 shows a perspective view of yet another device.

### DETAILED DESCRIPTION

It will be appreciated that the following description is intended to refer to specific examples of structure selected for illustration in the drawings and is not intended to define or limit the disclosure, other than in the appended claims.

We provide haemostatic devices for minimally invasive surgery, especially that performed in the abdominal cavity, which haemostatic devices are in the form of an absorbent disc made of a biodegradable porous material and having a center and a radial extension of at least about 10 mm from the center, which disc is formed in such a way that it can be converted into a state with a radius that is shortened in a substantially rotation-symmetrical way, in which state it can be inserted into the body with the aid of a tube, and from which state it can be converted back into the flat form again.

We provide flat pieces of a haemostatic material with a surface area that is sufficiently large for the wound to be treated, and where the conversion of the flat material enables release from a trocar in a simple way for easy placement on the wound.

The maximum diameter of the disc is therefore greater than the inside diameter of the trocar to be used in any given case, so that the disc can be released from the trocar in a simple way, due to its temporary form, especially one obtained by folding. In particular, this form is especially substantially rotation-symmetrical about the center of the disc. In a preferred case, the intermediate form is cup-shaped or funnel-shaped.

The diameter of the disc is generally in the range of about 20 to about 60 mm and especially about 30 to about 50 mm. Discs with a diameter of about 40 mm are suitable for normal use. The disc can have a circular circumference, but it can also be non-circular. A hexagonal outer contour is preferred when a number of discs are to be placed together basically without gaps between them. This can also be done with discs having an oval, triangular or rectangular shape, but polygonal shapes are also possible. The term “substantially rotation-symmetrical” refers to non-circular disc shapes and also to the folding at the outside edge.

In a preferred form, the disc has a substantially radial set of lines that favors conversion of the disc into a radially reduced form, and especially into a cup-shaped or funnel-shaped state. These lines can be structurings, patterns or profiles, preferably embossed lines. The disc-shaped material can be easily folded along these embossed lines. The set of lines can also be produced by folding already at this stage. For example, in a preferred structure, the disc has the shape of a piece of fluted filter paper. It is also possible to reverse the longitudinal direction of folding to produce double or multiple folds.

In another structure, the lines are produced by incision. These incisions, especially radial incisions, make it possible to convert the disc into a cup-like shape in a simple manner. The lines can extend from the outer edge of the disc to a point about 5 to about 10 mm from the center of the disc. This structure is preferred especially when the linear structures are incisions so that the center of the disc is retained as a flat center. If the lines are produced by embossment or folding, they can advantageously extend right to the center or to a point near it.

The center itself can be either closed or it has holes in it. This depends on how, i.e., with what means, the disc is pushed through a sleeve, especially a trocar. A center with holes or slits is preferred especially when several or many discs are arranged in the sleeve one behind the other and are penetrated by a feed rod that works on the individual discs in succession, with the discs facing the wound being released first.

The number of lines may depend on the nature of the set of lines in question. For example, preferably about 4 to about 20, especially about 6 to about 12 lines are possible especially when the lines are in the form of incisions. In this case, the discs can resemble opening petals when they are in the cup-shaped state. There can be about 3 to about 40 and especially about 3 to about 24 lines especially when they are in the form of embossments or folds. In the reduced state, the discs then have a pleated structure or the shape of a piece of fluted filter paper.

The disc generally has a surface area of about 3 to about 30 cm², preferably about 5 to about 15 cm² and especially about 8 to about 12 cm². In this range, the discs are still manageable and can cover sufficiently large wounds at the same time. The thickness of the discs normally vanes from about 1 to about 5 mm and especially from about 2 to about 3 mm. Thicker discs are also possible, partly because the material used for making them can be compressed, especially elastically, owing to its high porosity. Discs with a large diameter tend to have a smaller thickness. The thickness can also decrease from the center outwards.

The material used for making the discs is a protein and especially collagen or gelatin as in the well-known case. However, other biodegradable materials can also be employed. For example, the disc can be made of at least one polysaccharide, especially at least one chosen from a group comprising chitosan, dextran, hyaluronic acid, cellulose, oxidized cellulose and carboxymethylcellulose. The material forming the discs can be fibrous, especially when it is a polysaccharide. Textiles, especially woven and knitted types, also belong to this group.

The discs can be made of a single biodegradable material or from a mixture of two or more such materials. A layered structure, built up of at least two different and/or differently structured biodegradable materials, is another possibility for the haemostatic device. For example, one layer may include a protein, and the other at least one polysaccharide. A structure built up of different proteins, e.g., collagen and gelatin, is also feasible. One of the layers can be colored with the aid of a dye such as, for example, riboflavin or methylene blue to distinguish the layers from each other.

The porous material from which the haemostatic device is made advantageously has open pores. This enables it to absorb body fluids quickly. The porous material can also be compressed, especially in a reversible way, so that it returns to its original layer thickness on contact with a liquid.

In preferred structures, the haemostatic device is lyophilized (freeze-dried). Owing to freeze-drying and especially the choice of the solid content of the solutions or dispersions subjected to freeze-drying, the specific weight (the weight per cm²), the pore volume and the pore size can be fixed or influenced. The solid content of the solutions or dispersions, especially their collagen content, is preferably about 1 to about 5 wt %, calculated on the total weight of the solutions or dispersions. The density of the porous material is preferably about 10 to about 50 g/dm³, especially about 20 to about 40 g/dm³ and more preferably about 25 to about 35 g/dm³. The porous material from which the haemostatic device is made advantageously has a pore volume of over 90 vol % and especially one of about 96 to about 99 vol %. Its pore volume is generally about 96 to about 98 vol %. The specific weight of the porous material is normally about 5 to about 40 mg/cm², preferably about 10 to about 20 mg/cm² and especially about 10 mg/cm². The specific weight depends on how thick the layers of the material are.

If a lower pore volume is desired, the amount of biodegradable material in the solution or dispersion that may be subjected to freeze-drying is raised as much as possible. Compression, mentioned above, is an alternative. For example, the pore volume can be adjusted to about 60 to about 95 vol % by compression.

The porous material of the haemostatic device is supple and flexible despite being light. It is preferably stable up to a force of about 8 to about 15 newton and especially about 10 to about 13 newton when subjected to testing by a compression device operating with a stamping tool.

The haemostatic device can absorb an enormous amount of liquid. This amount is preferably about 15 to about 60 times, especially about 25 to about 60 times, and preferably about 30 to about 60 times its own weight. The high absorption capacity for liquids enables it to take up a large amount of liquid. In addition, the binding of body fluids occurs very quickly. For example, the porous material of the haemostatic device can be completely wetted with water in less than about 100 seconds and especially less than about 60 seconds. The situation is similar in the case of body fluids.

It is particularly advantageous to incorporate only one kind of protein in the haemostatic device, so that the latter does not contain other proteins. The preferred protein is collagen. Collagen can be present either in one form or as different kinds, called types I-IV.

In a preferred case, xenogenic collagen is used, especially porcine, bovine or equine collagen.

In a particularly advantageous case, the haemostatic device can also contain at least one organic acid, preferably at least one polyhydric organic acid, in the porous material. On fleeze-drying, such acids impart a membrane or platelet structure to the biodegradable material, which enlarges its internal surface area.

In a preferred form, the haemostatic device has a pH of less than about 4 in water or on contact with water. The pH of the haemostatic device in water or on contact with water is preferably about 3.0 to about 3.5.

The pore size of the haemostatic device is preferably below about 500 μm, especially under about 300 μm and preferably below about 100 μm. Owing to the small pore size, stronger capillary forces come into operation, so that the material can absorb more liquid, especially body fluids, and preferably blood.

In another form, the haemostatic device possesses flexible and especially stable properties. The acid content of the haemostatic device is preferably so high that these properties are not diminished. The acid content of the haemostatic device is preferably about 1 to about 25 wt %, especially about 2 to about 15 wt % and preferably about 3 to about 10 wt %.

In a further form, the water-soluble biocompatible organic acid is a non-volatile acid. It is preferably an aliphatic acid with for example 1-6 carbon atoms and preferably 3-6 carbon atoms in the chain. It can be an oligofunctional and especially a difunctional or trifunctional acid. It is especially a polyhydric acid, e.g., a dihydric acid. Preferably, the acid is a hydroxylcarboxylic acid, in particular, a polyhydric hydroxycarboxylic acid. It can also be a sugar acid, for example.

The water-soluble biocompatible organic acid present in the haemostatic device is preferably an acid chosen from the group comprising citric, tartaric, ascorbic, malic, gluconic, mucic, glutaric and adipic acid, for example. Malic acid is preferred in particular.

The water-soluble biocompatible organic acid in the haemostatic device can be pre-sent in particular in the form of a salt. This is preferably a calcium salt of the acid, e.g., calcium citrate. The use of a calcium salt is especially advantageous, since calcium ions can accelerate haemostasis.

We also provide haemostatic devices in the packed form. In this case, the disc is preferably already in the radially reduced form, especially in the cup-shaped or the funnel-shaped form. In other words, the haemostatic device, in particular in a radially reduced form, may be stored in a package. A disposable pack can be used here. The pack is preferably sterile. A sleeve-like pack is envisaged in particular, which can have an inside diameter of about 5 to about 13 mm. This sleeve, which is also called a reduction sleeve, can accommodate at least one disc. The discs can also be present in the form of an axial series of numerous radially reduced discs placed one behind the other. The pack can be a sleeve for a trocar. The sleeve may be such that it can be pushed into a trocar. The sleeve can also be so formed that its minimum content of one disc can be easily transferred from the sleeve into a trocar. Multiple discs in a radially reduced state may be stored in an axial array in a dispensing sleeve, the latter preferably being provided with an applicator for release of individual discs.

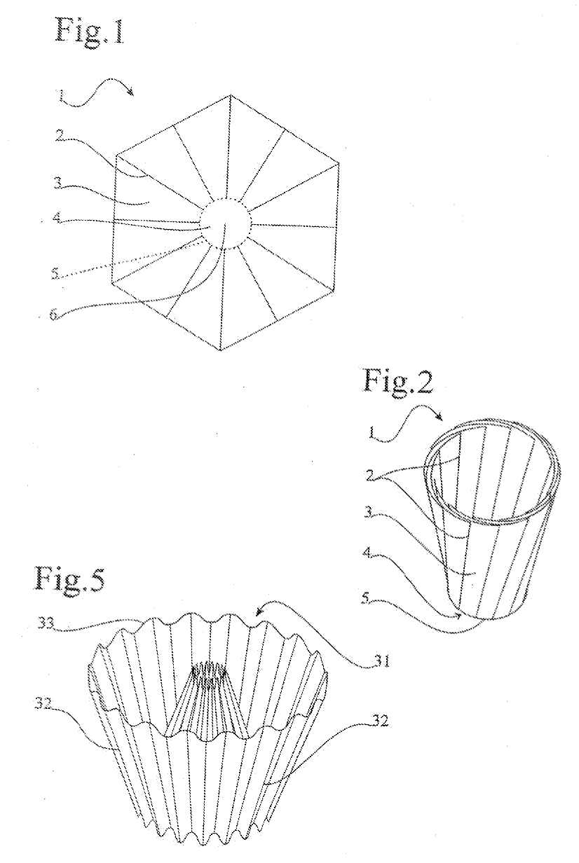

Turning now to the drawings, a haemostatic device 1 shown in FIGS. 1 and 2 is in the form of a hexagonal disc having a maximum diameter of 50 mm and a thickness of 2 mm. The disc comprises lyophilized collagen. The disc 1 is divided into twelve segments 3 by twelve radial lines 2. These radial lines are incisions that extend from the apices of the hexagon and from points in between these apices to a central region 4 of the disc 1, which region has a diameter of about 8 mm and lies in the center 6.

The lyophilized collagen material forming the disc 1 is soft and flexible and can be easily bent. Where the central region 4 turns into the segments 3, there is a contour line 5 for the central region, which line is embossed. The haemostatic device therefore looks like a composite flower with petals arranged around the central part. During use, the region 4 makes it easier to fix the disc 1 to the surface of the tissues, e.g., with a rod-like instrument or a feed rod.

As can be seen from FIG. 2, the segments 3 are bent in the same direction along the contour line 5 and overlap on one another at the outer edge. The haemostatic device therefore looks like a cup or an opening flower. In this state, the effective outside diameter of the haemostatic device is about 20 mm, but this diameter can be further reduced when the device is inserted into a tube with an inside diameter of about 10 mm. In this way, the haemostatic device can be used for haemostasis in the case of minimally invasive surgery by pushing it through a trocar, after which the segments return to the flat form without any problems. Fairly large surfaces can be covered without any gaps by placing a number of hexagonal discs one next to the other.

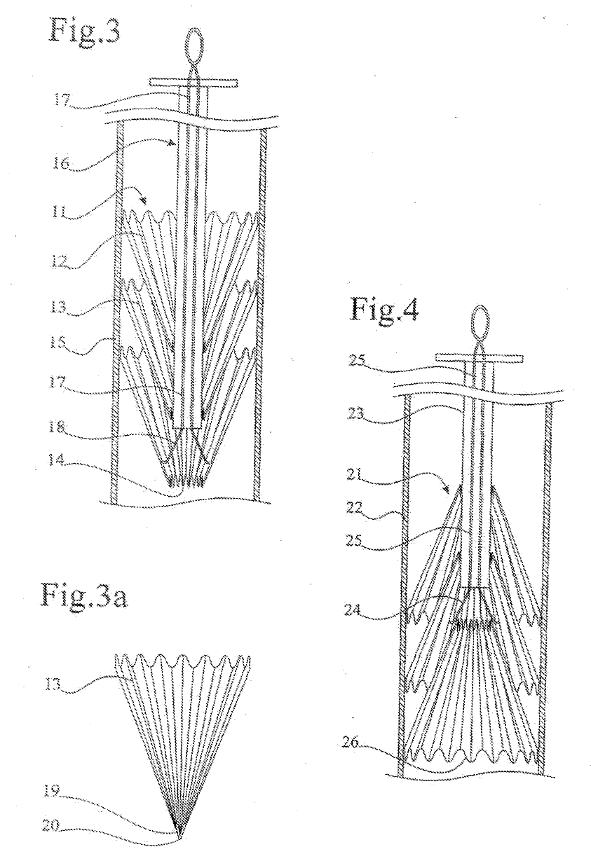

In the case of the structure shown in FIG. 3, circular discs 11 with a diameter of 40 mm are formed by folding to obtain an object shaped like an umbrella or a fluted filter paper. For this purpose, the discs have embossed radial lines 12 along which the lyophilized collagen material is folded alternately forward and backward. Thanks to the resulting folds 13, pushing the haemostatic devices together leads to a great reduction in the outside diameter of the disc, but FIG. 3 only shows part of this process for the sake of clarity. The tip of the folded disc (not shown) is cut off, so that an axial through-hole 14 is present at the center. Instead of this hole, there can also be short radial slits 20 at the center 19, e.g., cross-slits, so that no material is lost (see FIG. 3a).

In the structure shown here, there are three folded haemostatic devices arranged in the axial direction one behind the other in a tube 15, these haemostatic devices being partially pushed into one another. An applicator 16, which ends inside the haemostatic device that is in front, is pushed through the holes 14. The applicator 16 has a tubular shape. The applicator contains inside it a push rod 17 that has three radially disposed spring arms 18 at its leading end. These spring arms lie inside the folded disc, so that the latter can be pushed out of the tube 15 by the push rod 17. If the tube 15 is a trocar or a cylindrical holder that can be inserted into a trocar, then the haemostatic devices can be released in this way one after the other through the abdominal wall and onto the wound whose bleeding is to be stopped.

When the first haemostatic device has been released, the push rod 17 is withdrawn into the applicator 16, taking the spring arms 18 with it, these spring arms lying on one another. The applicator is then withdrawn a short distance so that its end comes to lie on the second haemostatic device which is now in the front position. The push rod is then again pushed out of the applicator with the spring arms, whereby the application procedure is repeated. Further haemostatic devices can be released in the same way after dispensing the second one.

The circular discs 21 illustrated in FIG. 4 are shaped in the same way as those shown in FIG. 3. However, they are inserted into a cylindrical holder 22 in the opposite direction. An applicator 23 is provided again, which is like the one used in FIG. 3. However, the spring arms 24 of a push rod 25 do not engage on the inside as in FIG. 3, but on the outside of the conically tapering region of the folded discs. The spring arms can again push out the folded disc from the cylindrical holder 22, but the folded outer edge of the disc 21 is the first to emerge from the tube. Again, any number of haemostatic devices can be kept in the cylindrical holder. The forvard movement of the applicator 23 is the same here.

As shown in FIGS. 3 and 4, the cylindrical holder, the haemostatic discs and the applicator can each be packed in sterile disposable packs that are opened when haemostasis is called for in minimally invasive surgery.

In FIG. 5, the haemostatic device is in the form of a twice folded disc 31. This disc has a base fold that corresponds to the structures shown in FIGS. 3 and 4. Radial embossments are again provided for forming the fold. The outer edge 33 of the disc is turned in the opposite direction at approximately the middle of the radial embossments so that two folds are obtained in the disc, one above the other. The folding is therefore similar to that of an umbrella that can be collapsed to make it shorter.

### EXAMPLE 1

33 g of collagen were made to swell in 660 ml of purest water (MilliQ water from the Millipor Company, Germany). The swollen collagen was suspended for about 20 minutes in a solvent mixture consisting of 1155 ml of purest water and 165 ml of isopropanol. About 1.32 g of malic acid were then dissolved in about 1320 ml of the suspension liquid, this amount representing 0.1 wt % of the total weight of the collagen suspension. Portions of 130 g of the suspension were then transferred into freeze-drying dishes with a base area measuring about 165 cm², and their contents were frozen at −40° C. and lyophilized. The product was obtained in the form of rectangular collagen plates. The thickness of the resulting plates varied with the amount of material in the freeze-drying dishes. The plates are normally made with a layer thickness of 1-2 mm, but thicker plates can be reduced to about half their thickness by compression. The compression can be coupled with the formation of embossed or punched radial lines, and the external form of the discs can be obtained in the required form and size by means of punching. Central holes or central radial slits can be formed at the same time. The required lines can also be formed without allowing for compression, and in the case of embossed lines a fold can be made at the same time.

### EXAMPLE 2

### Minimally Invasive Haemostasis in a Pig

Access to the spleen was produced in a minimally invasive manner with the aid of a trocar having an inside diameter of 10 mm. Capsular bleeding was induced in the spleen over an area of 2×2 cm. The trocar was used to apply circular collagen pads on the haemorrhaging wound, the pads having a diameter of 10 mm and a thickness of 4 mm. The bleeding could only be stopped after 10 minutes, despite the application of several layers. However, the bleeding could be stopped within 90 seconds when a circular cup-shaped haemostatic device was applied to a similar capsular lesion in the spleen, this device having an outer diameter of about 30 mm and six 10-mm incisions. The thickness of the collagen material was 4 mm.

Capsular bleeding was also induced in the liver after the creation of an access measuring 2×2 cm, using the minimally invasive technique. Both collagen pads and collagen cups with added malic acid, made as described in Example 1, were used in this experiment. The circular collagen pads, which had a diameter of 10 mm and a thickness of 4 mm, could not stop the bleeding within 10 minutes despite the application of several layers, but the application of the circular cups with an outside diameter of about 30 mm and with six 10-mm incisions managed to do this within 180 seconds in the case of a similar hepatic lesion.

1. A haemostatic device for minimally invasive surgery comprising: an absorbent disc made of a biodegradable porous material with a center and a radial extension of at least about 10 mm from the center, the disc formed to be converted into a radially reduced intermediate state with an essentially rotation-symmetrically shortened radius, in which state it can be introduced into a body through a tube, from which it can be released in a flat form.

2. The haemostatic device according to claim 1, wherein the disc has a set of sub-stantially radial lines that facilitate its conversion into a radially tapering state.

3. The haemostatic device according to claim 2, wherein the lines are embossments, folds or incisions.

4. The haemostatic device according to claim 2, wherein the lines extend from an outer edge of the disc to a point about 5 to about 10 mm from the center of the disc.

5. The haemostatic device according to claim 3, wherein the embossments and folds substantially extend to the center of the disc, with the proviso that the incisions end before reaching the center.

6. The haemostatic device according to claim 1, wherein the disc has a substantially circular circumference.

7. The haemostatic device according to claim 1, wherein the disc has a substantially polygonal circumference.

8. The haemostatic device according to claim 7, wherein the disc has a substantially tniangular, rectangular or hexagonal circumferential line.

9. The haemostatic device according to claim 1, wherein the disc has a central hole or a central slit.

10. The haemostatic device according to claim 2, wherein 4-20 lines are provided in the form of incisions.

11. The haemostatic device according to claim 2, wherein 3-40 lines are provided in the form of embossments or folds.

12. The haemostatic device according to claim 1, wherein the disc has a surface area of about 3 to about 30 cm².

13. The haemostatic device according to claim 1, wherein the disc has a thickness of about 1 to about 5.

14. The haemostatic device according to claim 1, formed from at least one protein.

15. The haemostatic device according to claim 1, formed from at least one polysaccharide selected from the group consisting of chitosan, hyaluronic acid, dextran, cellulose, oxidized cellulose and carboxymethylcellulose.

16. The haemostatic device according to claim 1, having a layered structure formed by at least two biodegradable materials.

17. The haemostatic device according to claim 1, wherein the disc comprises porous material having open pores.

18. The haemostatic device according to claim 1, wherein the disc comprises porous material which is compressed.

19. The haemostatic device according to claim 1, wherein the disc comprises porous material having a density of about 10 to about 50 g/dm³.

20. The haemostatic device according to claim 1, wherein the disc comprises porous material having a specific weight of about 5 to about 40 mg/cm².

21. The haemostatic device according to claim 1, having a pore volume of >90 vol %.

22. The haemostatic device according to claim 1, having a liquid absorption capacity corresponding to about 15 to about 60 times its own weight.

23. The haemostatic device according to claim 1, which can be completely wetted with water in <100 seconds.

24. The haemostatic device according to claim 1, which is freeze-dried.

25. The haemostatic device according to claim 1, further comprising at least one organic acid.

26. The haemostatic device according to claim 25, containing an acid having a pH of <4 when it is in contact with water.

27. The haemostatic device according to claim 25, wherein the acid is a hydroxy-acid.

28. The haemostatic device according to claim 1, further comprising a pack into which the disc is inserted.

29. The haemostatic device according to claim 28, wherein the pack is a disposable pack.

30. The haemostatic device according to claim 28, wherein the pack is a sterile pack.

31. The haemostatic device according to claim 28 wherein a plurality of discs are kept in the radially reduced state in an axial arrangement in a dispensing sleeve that is fitted with an applicator that releases the discs individually.
